(19) United States
(12) Patent Application Publication
(10) Pub. No.: US 2010/0316998 A1
(43) Pub. Date: Dec. 16, 2010

(54) METHODS AND COMPOSITIONS FOR EMPIRICAL SELECTION OF DRUGS OR OTHER MOLECULES

(75) Inventor: Malcolm L. Gefter, Lincoln, MA (US)

Correspondence Address: WOLF GREENFIELD & SACKS, P.C., 600 ATLANTIC AVENUE, BOSTON, MA, 02210-2206, US

(73) Assignee: Massachusetts Institute of Technology, Cambridge, MA (US)

(21) Appl. No.: 12/484,967

(22) Filed: Jun. 15, 2009

(51) Int. Cl. C12Q 1/68; C07H 21/04

(52) U.S. Cl. 435/6; 536/23.1; 536/24.33

(57) ABSTRACT

Methods for identification and/or selection of species having activity for a target molecule are described, as well as related compounds. In some cases, the compounds and methods may be useful in a screening process, where species that exhibit a desired property or combination of properties are identified and isolated from a large library of species (e.g., 10⁹ different species). The compounds may include an identification group and one or more binding moieties capable of interacting with a target molecule. Compounds and methods described herein may be useful in various applications such as drug discovery.

### FIELD OF THE INVENTION

The present invention relates to compositions and methods for drug discovery using combinatorial chemistry.

### BACKGROUND OF THE INVENTION

Combinatorial chemistry may be potentially useful in drug discovery, where high-throughput screening (HTS) of large libraries of chemical compounds are evaluated for their ability to interact with a particular biological target. For example, the biological target may be a protein, and compounds may be tested for their ability to inhibit or promote activity of the protein. In some cases, the compounds are evaluated for their ability to specifically interact with a biological target. For example, some drug selection technologies are based on the vertebrate humeral immune system, which is capable of synthesizing ligands (e.g., antibodies) that are specific and active for essentially any biological target. This selection process can be accomplished through the existence of a large (e.g., 10¹¹) and diverse library of natural antibodies, which can be synthesized by combinatorial assembly of structural components, random generation of additional diversity, followed by selection of effective binders.

In practice, many combinatorial methods are limited by the large number of compounds in the library and the inability of current methods to sufficiently identify active compounds within the library. Typically, a high degree of background “noise” exists due to the large number of compounds and related derivatives synthesized (e.g., 10⁹), making it challenging to detect a small number of compounds having activity for a particular target molecule. Such background “noise” may be generated by, for example, undesired dissociation of active compounds from the target molecule, as well as random binding of non-active compounds to the target molecule. Additionally, the methods typically include iterative fractionation of the library via numerous selection rounds to produce a small subset of compounds that can be individually synthesized and evaluated for the desired activity. However, with each fractionation or wash step, a large amount of signal is often lost due to the inadvertent removal of active compounds, such as those that have dissociated from the target molecule. Often, a significant portion of the signal is lost such that the number of molecules that can physically be detected falls below the level of detection required to successfully perform a polymerase chain reaction (PCR) (e.g., 10⁴ molecules) to enrich the active species. Thus, existing methods can often be impractical and time-consuming due to the iterative rounds of selection and significant loss of detectable signal.

Furthermore, libraries typically include families of related compounds which interact with the target molecule to varying degrees. For example, a library may contain hundreds of different compounds having related chemical structures, which each have sufficient interaction with the target molecule to collectively produce a detectable signal. However, while the interaction of target molecules with a large number of compounds may facilitate in increasing the signal-to-noise ratio of the library, subsequent identification, synthesis, and evaluation of each compound can be difficult and time-consuming.

### SUMMARY OF THE INVENTION

The present invention provides various compositions and methods useful in applications including selection of species from a large library of species, and/or other combinatorial methods. In some embodiments, the present invention relates to compositions comprising:

a compound having the structure,

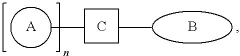

wherein:

A comprises at least one binding moiety capable of binding a target molecule;

B comprises an identification nucleotide group; and

C is a linker group tethered to A and B; and

n is at least 2,

wherein, in the presence of a target molecule, the compound can bind at least two target molecules.

In some embodiments, the present invention also provides compositions comprising:

a compound having the formula,

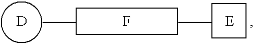

wherein:

D comprises at least one binding moiety capable of binding a first portion of a target molecule;

E comprises at least one binding moiety capable of binding or forming a bond with a second portion of the target molecule; and

F is tethered to D and E and is a linker group comprising a nucleotide group,

wherein, in the presence of the target molecule, D binds to the first portion of the target molecule and E binds to a second portion of the target molecule to form cyclic structure comprising the compound and the target molecule.

The present invention also provides methods comprising: providing a plurality of compounds comprising at least one active compound, each compound comprising at least one binding moiety and an identification nucleotide group tethered to the binding moiety, wherein the identification nucleotide group comprises a nucleotide sequence that is not a complement of and does not substantially interact with a nucleotide group of an adjacent compound; exposing the plurality of compounds to a target molecule such that the at least one active compound binds the target molecule to form a bound species comprising the at least one active compound and the target molecule; isolating the bound species; and increasing the amount of the active compound by amplifying the identification nucleotide group of the bound species via a polymerase chain reaction.

### BRIEF DESCRIPTION OF THE DRAWINGS

FIG. 1 shows a schematic representation of the interaction between a target molecule and a bivalent compound, according to one embodiment of the invention.

FIG. 2 shows a schematic representation of the interaction between a target molecule and a compound comprising a binding moiety and a crosslinker, according to one embodiment of the invention.

FIG. 3 shows a schematic representation of the synthesis of a library of compounds, according to one embodiment of the invention.

Other aspects, embodiments and features of the invention will become apparent from the following detailed description when considered in conjunction with the accompanying drawings. The accompanying figures are schematic and are not intended to be drawn to scale. For purposes of clarity, not every component is labeled in every figure, nor is every component of each embodiment of the invention shown where illustration is not necessary to allow those of ordinary skill in the art to understand the invention. All patent applications and patents incorporated herein by reference are incorporated by reference in their entirety. In case of conflict, the present specification, including definitions, will control.

### DETAILED DESCRIPTION

Methods for identification and/or selection of species having activity for a target molecule are described, as well as related compounds.

In some cases, the compounds and methods described herein may be useful in a screening process, where species that exhibit a desired property or combination of properties are identified and isolated from a large library of species (e.g., 10⁹ different species). Some embodiments may facilitate the identification of certain species within a library due to enhanced binding to a target molecule and/or a decrease in the background noise of the library. In some cases, species can be identified and isolated in fewer steps (e.g., fewer selection rounds) relative to known methods, resulting in recovery of larger amounts of the species. Compounds and methods described herein may be useful, for example, in drug discovery, where screening of a large number of compounds for “active” species that preferentially interact with a target molecule (e.g., biological molecule) can provide useful information in development of a drug.

Generally, embodiments described herein relate to the screening of a large number of compounds (e.g.,. a library) to determine which compound(s), if any, exhibit activity for a target molecule. A compound may be considered to exhibit activity for a target molecule by interacting with (e.g., binding to) the target molecule to a different or greater extent, relative to other compounds of the library and/or relative to other target molecules. Some embodiments of the invention may involve the combinatorial synthesis of a library of compounds, where each individual compound is attached to or “tagged” with an identification group, i.e., a moiety that uniquely identifies the compound. In one set of embodiments, the identification group may comprise a nucleotide group. The library may then be exposed to a target molecule (e.g., a biological target molecule) to allow for interaction between at least some compounds of the library and the target molecule, generating a determinable signal. Such active compounds may then be identified and isolated. In one set of embodiments, at least some compounds may bind the target molecule. The bound compound-target species may then be separated from the unbound species, and the identity of the bound species may be determined or “read” via the identification group. The activity of the compound may be further studied by additional screening and cross-screening methods. In some cases, the compound may provide information regarding the structure-activity relationship of an active compound, as described more fully below.

One advantageous feature of the invention relates to the ability to enhance formation and determination of the signal provided by active compounds of the library upon interaction with a target molecule. Relative to previous methods, where signal can be diminished due to undesired dissociation of active compounds from the target molecule and/or random binding of non-active compounds to the target molecule, embodiments disclosed herein may exhibit enhanced interaction (e.g., binding) with the target molecule to effectively maintain or “lock in” signal. In some embodiments, the effective binding constant of the compound may be enhanced and/or the “off-rate” of the compound may be decreased, as described more fully below.

Some embodiments described herein may also provide an improved ability to separate the bound species from the unbound species. As used herein, the term “bound species” refers to a species comprising a target molecule bound to a compound having activity for the target molecule, while the term “unbound species” refers to compounds which, upon exposure to a target molecule, exhibit low or substantially no activity for the target molecule and do not interact with the target molecule to form a bound species. In some embodiments, an active compound may have enhanced interaction with a target molecule, such that the bound species remains substantially intact during the screening process. That is, the active compound may have a decreased dissociation rate from the target molecule, i.e., may have a reduced “off-rate.” In some cases, compounds may have an increased affinity (e.g., binding constant) for a target molecule. Compounds having improved interaction with a target molecule to form a more robust or stable bound species may be more readily separated unbound species, thereby decreasing background “noise.” In some cases, the bound species may have a significant (e.g., 100-fold) difference in binding constant relative to the unbound species. For example, the compound may interact with a target molecule via formation of a bond, including a covalent bond. In some cases, the compound may interact with a target molecule via one or more locations of the compound. In some embodiments, a single compound may bind more than one target molecule, for example, via multiple binding moieties present on the compound. The compound may also bind one target molecule via multiple binding moieties on the compound.

In an illustrative embodiment, a compound may include a first binding moiety and a second binding moiety that each interact with target molecules immobilized on a solid support, thereby increasing the effective binding constant of the compound. That is, in the event that the first binding moiety dissociates from an immobilized target molecule (e.g., during fractionation or washing steps), the compound may remain attached to the solid support via the second binding moiety, which is bound to another immobilized target molecule, rather than diffuse away into solution. The first binding moiety may then reattach or “re-bind” to a target molecule immobilized on the solid support. In another illustrative embodiment, a compound may bind to a target molecule via at least one binding moiety, and the compound may also form a covalent bond to the bound target molecule, effectively “locking in” the target molecule.

Another advantageous feature of the invention involves the ability to synthesize libraries having increased signal-to-noise ratios, thereby enhancing the ability to identify active compounds. In some cases, the amount of bound species within a library may be increased (e.g., enriched) relative to other species at one or more stages during the screening process. For example, the bound species may be amplified after exposure of the library to a target molecule, but prior to fractionation of the library to isolate the bound species. That is, the amount of bound species may be increased relative to the amount of unbound species to reduce the amount of background noise present within library. This may allow for more facile separation of the bound species and may reduce the number of selection rounds needed to identify and isolate any active compounds. In some embodiments, the bound species may be amplified after a fractionation step. In some embodiments, amplification of the bound species is performed multiple times during the screening process.

Some embodiments provide compounds capable of interacting with (e.g., binding) a target molecule. In some cases, the compounds may interact with one or more target molecules via at least one binding moiety present within the compound. In some cases, the compounds comprise at least two binding moieties (e.g., bi-valent compounds). The binding moiety or moieties may interact with at least one binding site of a target molecule. The compound may also include other components, such as one or more identification group(s) which can be used to tag or identify the compound. In some embodiments, the identification group may be an identification nucleotide group such as a DNA oligonucleotide group. Compounds comprising multiple binding moieties for a target molecule (e.g., multi-valent compounds) in addition to an unique identification nucleotide group may have increased affinity for a target molecule, i.e., may have a decreased dissociation rate. Additionally, such compounds may be useful in creating a library having a high degree of diversity, wherein individual compounds can be readily separated from one another using the size and sequence of the identification nucleotide group (e.g., DNA identification group).

In some cases, the compound may also include a linker group tethered to the identification group and binding moiety or moieties. The linker group may comprise an atom or a group of atoms that are capable of forming chemical bonds to the identification group and binding moiety or moieties. In some cases, the linker group may be selected such that any number of components (e.g., binding moieties, identification groups, etc.) may be attached to the linker group. That is, the linker group may serve as the core structure present within compounds of a particular library. In some cases, the linker group may be selected such that various portions of the compound are positioned at a desired distance or location with respect to one another. For example, a compound may comprise two binding moieties which are arranged to have a certain distance from one another, as determined by the chemical structure and/or size of the linker group positioned therebetween. In some cases, the linker group may be selected to be compatible with conditions used in the combinatorial selection process. For example, the linker group may be selected such that it does not have substantial interaction with the target molecule and/or binding moieties of other compounds. In some embodiments, the linker may comprise an alkyl, heteroalkyl, alkenyl, heteroalkenyl, alknynyl, heteroalknynyl, aryl, heterocyclyl, or carbonyl group, any of which may be optionally substituted. In some embodiments, the linker may have a length of about 10-1,000 Angstroms, 25-500 Angstroms, 50-250 Angstroms, or, in some case, 50-150 Angstroms.

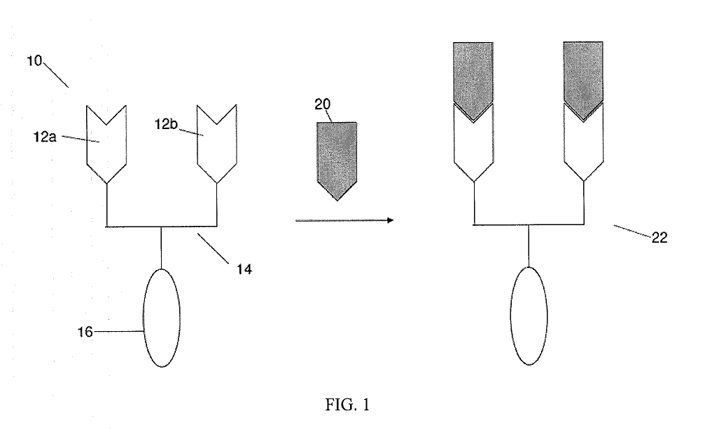

In one set of embodiments, the compound may have the structure,

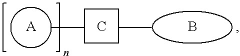

wherein A comprises at least one binding moiety capable of binding a target molecule; B comprises an identification group (e.g., identification nucleotide group); and C is a linker group tethered to A and B; and n is at least 2. In some embodiments, the compound can bind at least two target molecules, i.e., is a bi-valent compound. In some embodiments, n is greater than 2. FIG. 1 shows a schematic representation of a compound of the invention. Compound 10 includes linker 14 connected to binding moieties 12a and 12b. Linker 14 is also connected to identification group 16. During the screening process, compound 10 may be exposed to target molecule 20. Binding moieties 12a and 12b each interact with a target molecule to form bound species 22. Without wishing to be bound by theory, the bivalent binding behavior of the compound may be analogous to the behavior exhibited by bivalent antibody species.

In some embodiments, one or more identification group(s) may also be associated with at least one A group. In some embodiments, one or more identification group(s) may also be associated with each A group.

The compound may also include groups that are capable of interacting with different portions of a single target molecule. In some embodiments, the compound may include a group capable of interacting with a first portion of the target molecule, for example, via a binding interaction. The compound may also include a second, different group (e.g., a cross-linker) capable of interacting with a second portion of the target molecule via a binding interaction or formation of a bond. The first and second groups may be joined by a linker group that is selected to allow for interaction of the first and second groups with a target molecule, as described more fully below. Upon exposure to the target molecule, the groups may interact with the target molecule to form a bound species. In some cases, the bound structure may comprise a cyclized structure. For example, at least one of the first and second groups may form a covalent bond with a target molecule, which may effectively reduce or eliminate the dissociation rate or off-rate of the compound. This may facilitate isolation of the bound species and subsequent identification of the active compound. The compound may also include one or more identification groups associated with various portions of the compound.

In one set of embodiments, the compound may have the formula,

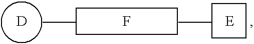

wherein D comprises at least one binding moiety capable of binding a first portion of a target molecule; E comprises at least one binding moiety capable of binding a second portion of the target molecule; and F is tethered to D and E and is a linker group comprising a nucleotide group (e.g., an identification nucleotide group). In some embodiments, D and/or F may be associated with one or more identification nucleotide group(s). For example, in the presence of the target molecule, D may bind to the first portion of the target molecule and E may bind to a second portion of the target molecule to form cyclic structure comprising the compound and the target molecule. One or more identification groups may optionally be associated with D, E, and/or F.

In some embodiments, E may comprise a group capable of forming at least one covalent bond with the target molecule (e.g., a cross-linker). For example, upon binding of the compound to a target molecule, the cross-linker (e.g., “E”) may be positioned in close enough proximity to the target molecule physically, such that E interacts with the bound target molecule to a greater extent, relative to other species present in solution. In some cases, the use of a cross-linker may reduce dissociation of the compound from the target molecule, i.e., may effectively reduce the off-rate of the compound. The cross-linker may be capable of forming a bond with the target molecule upon exposure to a chemical or biological species, electromagnetic radiation, a mechanical force, or, upon a change in temperature. The cross-linker may be capable of spontaneously forming a bond with the target molecule. In some cases, E may be a photoaffinity crosslinker. As used herein, the terms “photoaffinity crosslinker” or “photocrosslinker” refer to any moiety, or precursor thereof, that forms a covalent bond with a species upon exposure to electromagnetic radiation (e.g., UV light). For example, an azide group forms a reactive nitrene group upon exposure to electromagnetic radiation. Examples of photocrosslinkers include nitrenes, carbenes, ketones, cations, and radicals.

In some embodiments, the cross-linker may be a chemical cross-linker. That is, the cross-linker may comprise a functional group that is capable of interacting with a functional group on the target molecule, for example, via formation of a bond or via interaction between pairs of biological molecules. In some cases, the functional group may form a bond with the target molecule. The functional group may comprise an “electrophilic” atom, which refers to an atom which may be attacked by, and forms a new bond to, a nucleophile. In some cases, the electrophilic atom may comprise a suitable leaving group. The functional group may also be “nucleophilic” and may have a reactive pair of electrons. For example, the cross-linker may comprise a carbonyl group such as an aldehyde, an ester, a carboxylic acid, a ketone, an amide, an anhydride, or an acid chloride, a thiol, a hydroxyl group, an amine, a cyano group, charged moieties, or the like. In some embodiments, the cross-linker comprises an amine, a thiol, a carboxylic acid, an anhydride, or an alcohol.

In some cases, the cross-linker may be reacted via chemical reactions including substitution, condensation, metal-catalyzed coupling, halogenation, pericyclic reactions, other bond-forming reactions, and the like. In one set of embodiments, the cross-linker and the target molecule react via a 1,3-dipolarcycloaddition reaction, i.e., via “click chemistry.” For example, the cross-linker may comprise a dipolarophile that is reacted with a 1,3-dipolar compound present on the target molecule. Alternatively, the cross-linker may comprise a 1,3-dipolar compound that is reacted with a dipolarophile present on the target molecule. The 1,3-dipolar cycloaddition reaction may be performed under conditions that may be unreactive to the remainder of the compound (e.g., the carbon-based nanostructure), other than the dipolarophile. In an illustrative embodiment, a first species comprising a carbon-carbon triple bond may be reacted with a second species comprising an azide via a 1,3-dipolar cycloaddition to form a triazole ring that is covalently bound to the first and second species. Those of ordinary skill in the art would be able to select the appropriate reaction conditions and additives suitable for a particular 1,3-dipolar cycloaddition reaction. Methods for performing 1,3-dipolar cycloaddition reactions are also described, for example, in Synthetic Applications of 1,3-Dipolar Cycloaddition Chemistry Toward Heterocycles and Natural Products, A. Padwa, W. H. Pearson, Wiley-Interscience, 2002, the contents of which are incorporated herein by reference.

The linker (e.g., F) may be selected to have the appropriate length, flexibility, and/or chemical reactivity to allow both the first and second groups to interact with the target molecule. In some cases, the linker may also comprise an identification group, such as a identification nucleotide group. In some embodiments, the linker may be selected such that the cross-linker is able to form a bond (e.g., covalent bond) to the bound target molecule. For example, the linker may have the appropriate length and flexibility such that, upon interaction between the first group of the compound and the target molecule, the linker allow the second group to be positioned in sufficient, physical proximity to a different portion of the target molecule, such that the second group forms a covalent bond with the target molecule. In some embodiments, the second group may be positioned adjacent a portion of the target molecule comprising a group having complementary reactivity to the second group. The linker may comprise, for example, an alkyl, heteroalkyl, alkenyl, heteroalkenyl, alknynyl, heteroalknynyl, aryl, heterocyclyl, or carbonyl group, any of which may be optionally substituted. In some cases, the linker may comprise a nucleotide group. In some embodiments, the linker may have a length of about 10-1,000 Angstroms, 25-500 Angstroms, 50-250 Angstroms, or, in some case, 50-150 Angstroms. The linker group F may comprise an atom or a group of atoms that are capable of forming chemical bonds to the binding moiety D and cross-linker F. As described herein, the linker group may be selected to be compatible with conditions used in the combinatorial selection process, i.e., the linker does not have substantial interaction with the target molecule and/or binding moieties of compounds.

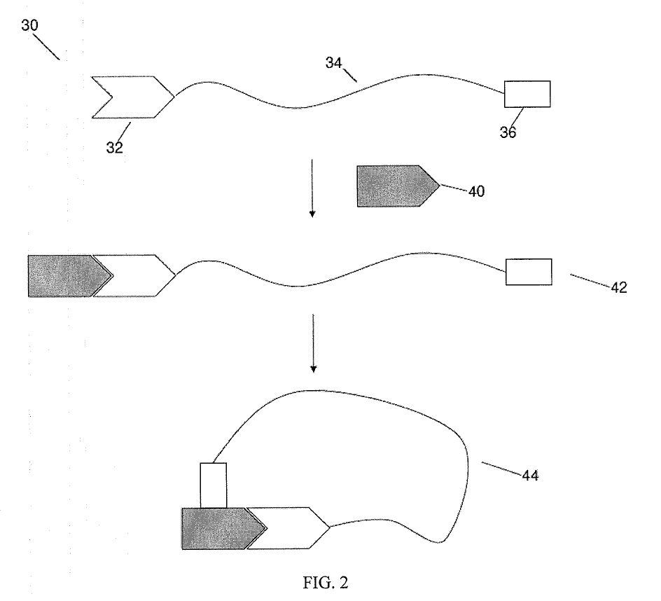

FIG. 2 shows a schematic representation of compound comprising a crosslinking group. Compound 30 includes a linker 34 tethered to a binding moiety 32 and a crosslinker 36. Upon exposure to target molecule 40, binding moiety 32 interacts with target molecule 40 to form bound species 42. Crosslinker 36 is then brought into sufficient proximity to bound target molecule 40 and forms a covalent bond to target molecule 40, thereby forming cyclized structure 44.

Embodiments described herein may be used alone or in combination to improve the combinatorial selection process. For example, a compound may be selected to include multiple binding moieties, an identification nucleotide, and at least one cross-linker.

Methods (e.g., combinatorial methods) for identifying a compound having activity for a particular target molecule are also provided. In some embodiments, the compound may be identified from a large library of compounds. For example, the method may involve providing a library of compounds as described herein, where each compound includes an identification nucleotide group. In some cases, the library may include at least 10², at least 10³, at least 10⁴, at least 10⁵, at least 10⁶, at least 10⁷, at least 10⁸, at least 10⁹, or greater, distinct, chemical compounds. In some embodiments, the library includes at least one, individual compound comprising at least two binding moieties and an identification nucleotide. In some embodiments, the library includes at least one, individual compound comprising at least one binding moiety, a cross-linker, and an identification nucleotide. In some cases, compounds of the library may be selected and designed to be capable of binding at least two target molecules. In some embodiments, the compound may be capable of binding a single target molecule via at least two binding moieties. The compounds may optionally include an identification group, such as an identification nucleotide group, that may be useful in uniquely identifying a particular compound within the library, as described more fully below.

The library of compounds may be provided in combination with one or more fluid carriers. In some cases, the library is provided as a solution, suspension, dispersion, or the like. In some embodiments, the library may be provided in association with a solid support, such as polymer beads, chromatography columns, and the like. In some cases, the library may be arranged on the surface of a solid substrate as an array of compounds, wherein the identity of each compound may be determined by its physical location in the array. Those of ordinary skill in the art would be able to select the appropriate method for providing the library of compounds.

The library may then be exposed to a target molecule using various methods known in the art, to allow any active compounds to interact with the target molecule. In some cases, at least one compound binds the target molecule via a binding moiety of the compound, thereby forming a bound species that includes the active compound and the target molecule. The interaction between the target molecule and the binding moiety may, in some embodiments, comprise formation of a bond, such as a covalent bond (e.g. carbon-carbon, carbon-oxygen, oxygen-silicon, sulfur-sulfur, phosphorus-nitrogen, carbon-nitrogen, metal-oxygen or other covalent bonds), an ionic bond, a hydrogen bond (e.g., between hydroxyl, amine, carboxyl, thiol and/or similar functional groups, for example), a dative bond (e.g. complexation or chelation between metal ions and monodentate or multidentate ligands), or the like. The interaction may also comprise Van der Waals interactions. In one embodiment, the interaction comprises formation of a covalent bond between the compound and the target molecule. In some cases, the binding moiety may be an electron-rich or electron-poor moiety within the compound, wherein interaction between the target molecule and the compound comprises an electrostatic interaction.

The compound may also be capable of biologically binding a target molecule via an interaction that occurs between pairs of biological molecules including proteins, nucleic acids, glycoproteins, carbohydrates, hormones, and the like. For example, the compound may comprise a binding moiety, such as biotin, that specifically binds to a complementary entity, such as avidin or streptavidin, on a target molecule. Other examples of pairs of biological molecules include an antibody/peptide pair, an antibody/antigen pair, an antibody fragment/antigen pair, an antibody/antigen fragment pair, an antibody fragment/antigen fragment pair, an antibody/hapten pair, an enzyme/substrate pair, an enzyme/inhibitor pair, an enzyme/cofactor pair, a protein/substrate pair, a nucleic acid/nucleic acid pair, a protein/nucleic acid pair, a peptide/peptide pair, a protein/protein pair, a small molecule/protein pair, a glutathione/GST pair, an anti-GFP/GFP fusion protein pair, a Myc/Max pair, a maltose/maltose binding protein pair, a carbohydrate/protein pair, a carbohydrate derivative/protein pair, a metal binding tag/metal/chelate, a peptide tag/metal ion-metal chelate pair, a peptide/NTA pair, a lectin/carbohydrate pair, a receptor/hormone pair, a receptor/effector pair, a complementary nucleic acid/nucleic acid pair, a ligand/cell surface receptor pair, a virus/ligand pair, a Protein A/antibody pair, a Protein G/antibody pair, a Protein L/antibody pair, an Fc receptor/antibody pair, a biotin/avidin pair, a biotin/streptavidin pair, a drug/target pair, a zinc finger/nucleic acid pair, a small molecule/peptide pair, a small molecule/protein pair, a small molecule/target pair, a carbohydrate/protein pair such as maltose/MBP (maltose binding protein), a small molecule/target pair, or a metal ion/chelating agent pair. In some embodiments, the compound may interact with (e.g., bind to) an active site of a target molecule, including the active site of an enzyme.

The nature of the interaction between a compound and a target molecule can vary depending on, for example, the energy state of the binding moiety or target molecule. In some cases, the compound may interact with the target molecule when the target molecule is in an excited state. Alternatively, the compound may interact with a target molecule when the compound, or portion thereof, is in an excited state. For example, a compound may interact with a target enzyme, such that the enzyme binds an excited state compound with higher affinity than a ground state compound. Compounds may be placed in an excited state by, for example, random fluctuations in energy, i.e., after collision with another molecule, or by exposure to an external source of energy, including electromagnetic, radioactive, mechanical, acoustic, or thermal energy.

In some cases, the non-covalent affinity of a binding moiety for a target molecule can be determined using an equilibrium constant such as the dissociation constant, K_{d}, or a related function such as the association constant Kₐ. In some embodiments, the dissociation constant for a binding moiety binding to a target molecule less than 1 mM. In some embodiments, the dissociation constant is less than 1 μM, less than 1 nM, less than 1 pM, less than 1 fM, less than 1 aM, or, in some cases, less than 1 zM.

In some embodiments, the compounds may be associated with a solid support, such polymer beads, a chromatography column, or other solid supports, and, upon exposure of the compounds to the target molecule, at least some active compounds may bind the target molecule, thereby immobilizing the target molecule on the solid support. In some embodiments, the compounds may be exposed to a solution comprising the target molecule. In some cases, the target molecule may be associated with a solid support, such that exposure of the target molecule to a solution containing the library allows for immobilization of any active compounds on the solid support. In some embodiments, the target molecule and library of compounds may be combined together in solution, i.e., in the absence of a solid support.

The bound species may then be separated or isolated from the unbound species, using various methods known in the art. For example, in cases where the bound species is immobilized on a solid support via either the target molecule or active compound, the bound species may be isolated by rinsing or washing the solid support in order to remove the unbound species. For example, the bound species may be immobilized on a chromatography column, such that any unbound species may be eluted by passing solvent through the column. In some cases, the bound species may be determined by the emission of a signal, such as a fluorescence signal, and subsequently isolated. For example, the compounds may be arranged in an array on a solid support, such that the bound species emit a signal (e.g., via a fluorescent tag) and the unbound species emit a different signal, a decreased signal, or substantially no signal. The bound species may then be isolated based on the location of the fluorescence emission. In cases where the target molecule and library are combined in solution, the bound species may be removed via filtration methods (e.g., gel filtration), affinity methods, and the like. In an illustrative embodiment, the target molecule may be a cell, and, upon binding of the target cell to at least some active compound(s) in the library, a non-target cell-type having a cell surface that is different from the target cell surface may be used to bind and remove the unbound species from the library.

The amount of the active compound may optionally be increased (e.g., amplified, enriched) by amplifying the identification nucleotide group of active compounds associated with the bound species (e.g., via a polymerase chain reaction). In some embodiments, each compound in a library may comprise a unique nucleotide sequence, such that a compound which exhibits activity for a target molecule (e.g., by forming a bound species with the target molecule) may be readily identified by the nucleotide sequence. For example, each compound may include a DNA oligonucleotide which does not substantially interact (e.g., cross-hybridize) with other DNA oligonucleotides present within the library. Additionally, the use of compounds having unique nucleotide sequences can allow for improved enrichment of the active compound and reduced enrichment of non-active species (e.g.,. unbound species). In some embodiments, the act of increasing the amount of the active compound is performed prior to the act of isolating the bound species. In some embodiments, the act of increasing the amount of the active compound is performed following the act of isolating the bound species. In some embodiments, the act of increasing the amount of the active compound is performed both prior to and following the act of isolating the bound species. For example, a DNA oligonucleotide of an active compound may be enriched such that its relative proportion within the library may be increased, in some cases, prior to a new round of selection.

Enrichment of the identification nucleotide sequence, and, thus, the active compound, may be performed using various methods known in the art. Some embodiments involve the use of a polymerase chain reaction (PCR) to amplify the bound species. The term “polymerase chain reaction” is given its ordinary meaning in the art and refers to the reaction by which copies of DNA fragments can be rapidly replicated. For example, the identification nucleotide group of the bound species may comprise a PCR primer sequence that may allow for interaction of the nucleotide group with a DNA polymerase. The design and synthesis of primer oligonucleotides is well-known in the art, including reaction conditions and reagents for performing the reaction. Examples of PCR primer sequences are described in, for example, Innis, et al., eds., PCR Protocols: A Guide to Methods and Applications, San Diego: Academic Press (1990), the contents of which are incorporated herein by reference in their entirety. Other amplification methods may also be used in the context of the invention, and those of skill in the art would be able to select methods suitable for use in a particular application. In some embodiments, a ligase chain reaction (LCR) may be used to amplify the bound species. In some cases, cell cloning techniques may be used.

The compounds and methods described herein provide the ability to screen a large library of candidate compounds to identify those compounds which exhibit a particular set of desired properties. In some embodiments, a compound may be selected for its activity and/or selectivity for a target molecule. In some embodiments, a compound is selected for it ability to interact primarily, or substantially exclusively, with a desired target molecule and its reduced activity relative to other target molecules. Such methods may be useful, for example, in identifying drug candidates for a target biological molecule.

Embodiments of the invention may be useful in providing information for the development of additional drug candidates. Upon isolation and identification of compound(s) which exhibit activity for a target molecule, a series of structurally related compound (e.g., analogs) may be synthesized and screened for potential activity. For example, several compounds which contain similar chemical features may be found to have a degree of activity. Using this information, a structure-activity relationship (SAR) may be formed in order to provide information regarding the features or characteristics of the compound which may affect activity. In some embodiments, the SAR may be useful in developing and/or improving drug candidates.

Additional screening tests may be conducted upon identification of active compound(s). For example, cross-screening tests may be conducted to determine whether the active compounds interact with other related target molecules to a degree that interferes with its ability to interact with the desired target molecule. Also, the active compound, lacking the identification nucleotide, may be evaluated in vitro or in vivo for its ability to interact with the target molecule.

In an illustrative embodiment, the method may comprise synthesis of a large number (e.g., library) of compounds using combinatorial methods, where each compound may be attached to a unique identification nucleotide. The library of compounds may be then be exposed to a target molecule (e.g., a biomolecule), allowing for interaction (e.g., binding) between the target molecule and compounds having activity for the target molecule, producing bound species. The bound species may then be separate from the unbound species, and the bound species are identified or “read” by their unique identification tag. The compounds which exhibit activity with respect to the target molecule may then by synthesized separately in order to confirm their activity by various in vitro or in vivo assays. Additionally, the active compounds may be cross-screened to determine toxicity and/or to evaluate other safety concerns.

Compounds described herein may be useful in combinatorial screening processes, as well as other applications. In some cases, the compounds may be useful in evaluating the activity of a compound with respect to a wide range of target molecules. For example, the ability to provide information regarding the effect of a compound on both desired target molecules and undesired target molecules may be advantageous in drug discovery. That is, the ability to not only evaluate the desired activity of a compound, but also predict any potential side effects caused by the compound, would provide a powerful tool in the selection of an effective drug. In some cases, a compound comprising at least one crosslinking group may be exposed to a series of target molecules to provide information regarding the interaction between the compound and various target molecules. In some cases, the compound may be exposed to a complete mixture or cytological preparation to identify target species within the proteome for which the compound may exhibit activity. Such information may be used to create a profile of a compound's effect on various target species (e.g., proteins) in the body, to identify potential activity and/or side effects of the compound.

In some embodiments, compounds may be useful in therapeutic or diagnostic applications. For example, a multi-valent compound (e.g., bi-valent compound) may be used as a therapeutic agent in place of an antibody, either alone or in combination with another species (e.g., a reactive protein species such as Fc portions of antibodies, enzymes, and the like). In another example, a compound may be useful as a diagnostic agent for a target molecule.

Compounds as described herein may comprise an identification group and at least one binding moiety, and may optionally comprise a linker and/or a cross-linker. In some embodiments, the identification group is a nucleotide, such as a DNA molecule. In some cases, the identification nucleotide group is a single-stranded DNA molecule. The nucleotide group may comprise a unique sequence of individual nucleotides linked together to form an oligonucleotide or a polynucleotide. The term “nucleotide” refers to any moiety capable of forming a Watson-Crick pair with another, complementary nucleotide, and can be replicated by DNA polymerases in a PCR reaction. The nucleotide group may comprise naturally occurring nucleotides or non-naturally occurring nucleotides. Examples of nucleotides include adenosine (A), guanosine (G), thymidine (T), and cytosine (C). It should be understood that other nucleotides or analogs thereof may be used in the context of the invention, including those described in U.S. Pat. No. 5,432,272, the contents of which are incorporated herein by reference.

Identification nucleotide groups may be synthesized using known methods, including enzyme catalysis (e.g.,. using DNA ligase). Those of ordinary skill in the art would be able to select the appropriate enzyme and conditions (e.g., temperature, solvent, pH) suitable for use in a particular application. In some cases, the identification nucleotide group comprises at least 2, at least 3, at least 5, at least 10, at least 25, at least 50, or, in some cases, at least 100 nucleotides. In some embodiments, the identification nucleotide group comprises a terminal sequence that can serve as a primer for PCR.

The compounds described herein may also include at least one binding moiety. In some cases, the compound comprises at least two or more binding moieties. As used herein, the term “binding moiety” refers to any atom, molecule, portion of a molecule, or precursor thereof, capable of having a binding interaction with a target molecule. In some cases, the binding moiety may comprise a biological or a chemical molecule able to bind to another biological or chemical molecule in a medium (e.g., solution, vapor phase, solid phase). For example, the binding moiety may be a thiol, aldehyde, ester, carboxylic acid, hydroxyl, sulfonyl, phosphonyl, epoxide, aziridine, isocyanate group, or the like, wherein the binding moiety forms a bond with the target molecule. In some cases, the binding moiety may be an electron-rich or electron-poor moiety, wherein interaction between the binding moiety and the target molecule comprises an electrostatic interaction. The binding moiety may also be capable of biologically binding a target molecule via an interaction that occurs between pairs of biological molecules, as described herein.

In some cases, the compound may comprise a binding moiety precursor. A binding moiety precursor refers to any atom or group having a reduced ability to interact with a target molecule, and may be modified (e.g., chemically modified) such that its ability to interact with a target molecule is increased. For example, the binding moiety precursor may be chemically modified prior to exposure to a target molecule. In some cases, modification of a binding moiety precursor may include formation of a bond between the binding moiety precursor and another atom or group. For example, two or more binding moiety precursors may be attached to form a single binding moiety. In some cases, modification of a binding moiety precursor may include cleavage of a bond between the binding moiety precursor and another atom or group. In some embodiments, modification of a binding moiety precursor may comprise both cleavage of a bond and formation of a bond.

Compounds of the invention may be synthesized using methods known in the art. Generally, the compounds may be produced by reacting one or more components (e.g., “building blocks”) having complementary reactive groups to produce a library of compounds. In some embodiments, parallel syntheses of various compounds may be conducted by reacting separate “batches” of compounds for addition of each building block. For example, identification nucleotide groups may be appended to different compound precursors in a first series of batches. The batches may then be pooled or combined, and additional components such as binding moieties may be appended to the compound precursors, for example, in separate ligation steps. Each additional component or building block may also be encoded by an identification group.

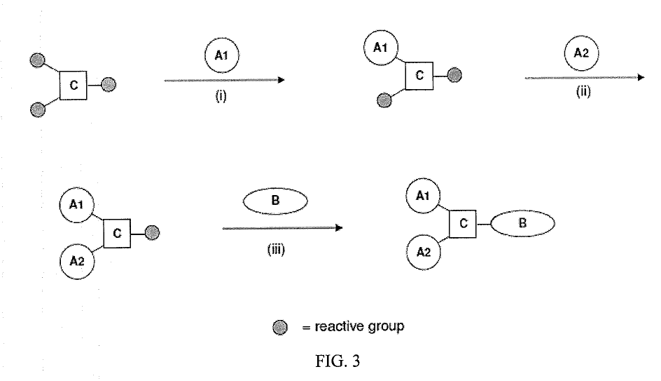

The synthesis of a library may involve reacting compound precursors that are capable of forming a bond with various components in a series of chemical reactions. For example, a compound precursor (or “core”) comprising multiple reactive groups may be reacted, in separate steps, with a series of components each having only one reactive group, such that the core structure is appended with the desired number and kind of components to produce the final compound. FIG. 3 shows an illustrative embodiment wherein compound precursor C comprises three reactive groups, indicated by gray circles. Compound precursor C may be reacted in a series of independent and separate reactions, including (i) addition of a first binding moiety A1, (ii) addition of a second binding moiety A2, and (iii) addition of an identification nucleotide group B. It should be understood the various components including linkers, cross-linkers, binding moieties, and/or identification groups may be reacted in any order and in any amount suitable for a particular application. Additionally, each component may include an identification group, such as a DNA nucleotide group having a defined sequence. Such methods may be useful in synthesizing libraries having a high degree of diversity, wherein the compounds are readily identifiable via one or more identification groups. Referring to FIG. 3, the use of 1000 different binding moieties for A1 in step (i), 1000 different binding moieties for A2 in step (ii), and 1000 different identification nucleotide groups for B in step (iii) may produce a library having a diversity of 10⁹ compounds.

Components of the compounds may be tethered to one another using chemical reactions known in the art, wherein the components are capable of reacting together in a manner that produces a desired chemical bond. Such reactions are known in the art, for example, in “Advanced Organic Chemistry” by Carey and Sundberg and in “Advanced Organic Chemistry” by Jerry March, the contents of which are incorporated herein by reference. Some specific examples of reactions that may be useful in synthesizing compounds described herein include substitution, elimination, condensation, aromatic substitution, pericyclic reactions, Wittig reactions, metal-catalyzed reactions, and the like. Chemical reactions may be selected to be compatible with (e.g., inert to) other functional groups that may be present during synthesis of the library and subsequent use of the library in, for example, a screening process. In some embodiments, the chemical reaction may be selected to be compatible with nucleotide groups. In some embodiments, protecting groups may be used to prevent reaction at sites other than the desired reactive site. The phrase “protecting group” as used herein refers to temporary substituents which protect a potentially reactive functional group from undesired chemical transformations. Examples of such protecting groups include esters of carboxylic acids, silyl ethers of alcohols, and acetals and ketals of aldehydes and ketones, respectively. The field of protecting group chemistry has been reviewed (Greene, T. W.; Wuts, P. G. M. Protective Groups in Organic Synthesis, 2^{nd} ed.; Wiley: New York, 1991). In some cases, protecting groups may impart other beneficial characteristics, including improved solubility of a compound in a particular solvent.

In one set of embodiments, formation of a bond may be performed by reaction of an amine group with various electrophilic groups. For example, reaction between a carbonyl group and an amine to form an amide. Similarly, reaction between a sulfonyl group or phosphonyl group with an amine to form, respectively, a sulfonamide group or a phosphonamidate group. In one set of embodiments, formation of a bond may be performed by reaction of a hydroxyl group with various electrophilic groups.

In some embodiments, synthesis of a compound or library of compounds may include formation of an oligomeric or polymeric chain (e.g., linear, branched, hyperbranched) by reacting monomers with at least two reactive groups. Examples of polymers include polyesters, polyamides, polycarbonates, and the like. Certain embodiments include synthesis of biopolymeric species such as polypeptides, polynucleotides, polysaccharides, or any combination of these species. The biopolymers may be linear, branched, or cyclic, and may comprise naturally-occurring and/or synthetic variants of the polymers. In some embodiments, synthesis of a compound or library of compounds involves separate synthesis of an oligonucleotide or polynucleotide, followed by attachment of the oligonucleotide or polynucleotide to a compound precursor. In some cases, the compound precursor may be coupled to a monomeric nucleotide or other reactive group to which a series of nucleotides may be iteratively coupled, forming an oligonucleotide or polynucleotide.

As used here, a “reactive group” refers to a portion of a molecule at which a chemical reaction can occur.

The concentrations of the reactants may be defined as being equimolar with respect to the reactant involved in the reaction. The concentration of one reactant may be greater than the concentration of another reactant to facilitate a better reaction yield of one reactant. In cases where not all of the building blocks have reacted, capping reagents may be used to react with the building block in order to prevent participation of the unreacted building block during a subsequent reaction step. Examples of capping reagents include, anhydrides for capping hydroxyl groups and amines as esters or amides, for example; alcohols for capping carboxylic acids as esters, for example; alkyl halides for capping hydroxyl groups as ethers and amines as alkyl amines, for example; disulfides for protecting thiols, for example; etc.

The invention also contemplates purification schemes for separating building blocks and functional groups from, for example, by products of reactions, unreacted reagents, solvents, etc.

Other methods for synthesizing libraries of compounds are described in Jung, G., Combinatorial chemistry: synthesis, analysis, screening (Wiley-VCH, 1999), the contents of which are incorporated herein by reference.

The target molecule may be any chemical or biological species. In some cases, the target molecule is a biological target molecule. For example, the target molecule may be an enzyme, such as a protein enzyme or nucleic acid enzyme (“ribozyme”). Other examples of target molecules include a protein receptor, an antibody or aptamer (e.g., polypeptides, polynucleotides, polysaccharides, or synthetic polymers such as peptide-nucleic acids). The monomeric units of polymers comprising the target molecule could be synthetic, for example 2′-fluoro-modified RNA or locked nucleic acid. The binding moiety may also bind to an effector site on a target molecule that changes the activity of the target molecule. For example, the binding of a binding moiety to an effector site may increase or decrease the catalytic activity of an enzyme towards a substrate. The specificity of the enzyme for a substrate may also be changed. In one embodiment, the enzyme may be able to act on an alternative substrate upon binding of an effector to the enzyme. In an alternative embodiment, a binding moiety binds to a target molecule in a manner that leaves the target molecule essentially unchanged with respect to the function of the target molecule.

The methods described herein may be conducted in the presence of one or more solvents or fluid carriers. The solvents may be aqueous, organic, inorganic, or any combination of these solvents and may contain at least one solute. Examples of aqueous solutions are distilled water and water containing at least one buffering species such as Tris buffer, phosphate buffer, HEPES buffer, or cacodylate buffer, ammonium acetate, triethylammonium acetate, triethylammonium bicarbonate, ammonium phosphate, etc. The buffer may also organic, inorganic, or a combination of organic and inorganic molecules. Aqueous solutions may also include at least one species of inorganic salt such sodium chloride or an organic salt such as tetrabutylammonium tosylate. In another embodiment, aqueous solutions could contain a neutral solute such as glucose. Solutions may be aqueous-organic, where water and a water-miscible organic solvent are mixed. Examples of water-miscible organic solvents include alcohols such as methanol, ethanol, and isopropanol, dimethylsulfoxide, acetonitrile, acetone, etc. Aqueous-organic solutions also can contain organic or inorganic solutes. The pH of aqueous and aqueous-organic solutions can be acidic, neutral, or basic.

In the compounds and compositions of the invention, the term “alkyl” refers to the radical of saturated aliphatic groups, including straight-chain alkyl groups, branched-chain alkyl groups, cycloalkyl (alicyclic) groups, alkyl substituted cycloalkyl groups, and cycloalkyl substituted alkyl groups. In some embodiments, a straight chain or branched chain alkyl may have 30 or fewer carbon atoms in its backbone, and, in some cases, 20 or fewer. In some embodiments, a straight chain or branched chain alkyl may have 12 or fewer carbon atoms in its backbone (e.g., C₁-C₁₂ for straight chain, C₃-C₁₂ for branched chain), 6 or fewer, or 4 or fewer. Likewise, cycloalkyls may have from 3-10 carbon atoms in their ring structure, or 5, 6 or 7 carbons in the ring structure. Examples of alkyl groups include, but are not limited to, methyl, ethyl, propyl, isopropyl, cyclopropyl, butyl, isobutyl, tert-butyl, cyclobutyl, hexyl, cyclochexyl, and the like.

The term “heteroalkyl” refers to an alkyl group as described herein in which one or more carbon atoms is replaced by a heteroatom. Suitable heteroatoms include oxygen, sulfur, nitrogen, phosphorus, and the like. Examples of heteroalkyl groups include, but are not limited to, alkoxy, amino, thioester, and the like.

The terms “alkenyl” and “alkynyl” refer to unsaturated aliphatic groups analogous in length and possible substitution to the alkyls described above, but that contain at least one double or triple bond respectively.

The terms “heteroalkenyl” and “heteroalkynyl” refer to unsaturated aliphatic groups analogous in length and possible substitution to the heteroalkyls described above, but that contain at least one double or triple bond respectively.

As used herein, the term “halogen” or “halide” designates —F, —Cl, —Br, or —I.

The terms “carboxyl group,” “carbonyl group,” and “acyl group” are recognized in the art and can include such moieties as can be represented by the general formula:

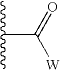

wherein W is H, OH, O-alkyl, O-alkenyl, or a salt thereof. Where W is O-alkyl, the formula represents an “ester.” Where W is OH, the formula represents a “carboxylic acid.” The term “carboxylate” refers to an anionic carboxyl group. In general, where the oxygen atom of the above formula is replaced by sulfur, the formula represents a “thiolcarbonyl” group. Where W is a S-alkyl, the formula represents a “thiolester.” Where W is SH, the formula represents a “thiolcarboxylic acid.” On the other hand, where W is alkyl, the above formula represents a “ketone” group. Where W is hydrogen, the above formula represents an “aldehyde” group.

The term “aryl” refers to aromatic carbocyclic groups, optionally substituted, having a single ring (e.g., phenyl), multiple rings (e.g., biphenyl), or multiple fused rings in which at least one is aromatic (e.g., 1,2,3,4-tetrahydronaphthyl, naphthyl, anthryl, or phenanthryl). That is, at least one ring may have a conjugated pi electron system, while other, adjoining rings can be cycloalkyls, cycloalkenyls, cycloalkynyls, aryls and/or heterocyclyls. The aryl group may be optionally substituted, as described herein. “Carbocyclic aryl groups” refer to aryl groups wherein the ring atoms on the aromatic ring are carbon atoms. Carbocyclic aryl groups include monocyclic carbocyclic aryl groups and polycyclic or fused compounds (e.g., two or more adjacent ring atoms are common to two adjoining rings) such as naphthyl groups. The term “heteroaryl” refers to aryl groups comprising at least one heteroatom as a ring atom.

The term “heterocycle” refers to cyclic groups containing at least one heteroatom as a ring atom, in some cases, 1 to 3 heteroatoms as ring atoms, with the remainder of the ring atoms being carbon atoms. Suitable heteroatoms include oxygen, sulfur, nitrogen, phosphorus, and the like. In some cases, the heterocycle may be 3- to 10-membered ring structures or 3- to 7-membered rings, whose ring structures include one to four heteroatoms. The term “heterocycle” may include heteroaryl groups, saturated heterocycles (e.g., cycloheteroalkyl) groups, or combinations thereof. The heterocycle may be a saturated molecule, or may comprise one or more double bonds. In some case, the heterocycle is a nitrogen heterocycle, wherein at least one ring comprises at least one nitrogen ring atom. The heterocycles may be fused to other rings to form a polycylic heterocycle. The heterocycle may also be fused to a spirocyclic group. In some cases, the heterocycle may be attached to a compound via a nitrogen or a carbon atom in the ring.

Heterocycles include, for example, thiophene, benzothiophene, thianthrene, furan, tetrahydrofuran, pyran, isobenzofuran, chromene, xanthene, phenoxathiin, pyrrole, dihydropyrrole, pyrrolidine, imidazole, pyrazole, pyrazine, isothiazole, isoxazole, pyridine, pyrazine, pyrimidine, pyridazine, indolizine, isoindole, indole, indazole, purine, quinolizine, isoquinoline, quinoline, phthalazine, naphthyridine, quinoxaline, quinazoline, cinnoline, pteridine, carbazole, carboline, triazole, tetrazole, oxazole, isoxazole, thiazole, isothiazole, phenanthridine, acridine, pyrimidine, phenanthroline, phenazine, phenarsazine, phenothiazine, furazan, phenoxazine, pyrrolidine, oxolane, thiolane, oxazole, oxazine, piperidine, homopiperidine (hexamnethyleneimine), piperazine (e.g., N-methyl piperazine), morpholine, lactones, lactams such as azetidinones and pyrrolidinones, sultams, sultones, other saturated and/or unsaturated derivatives thereof, and the like. The heterocyclic ring can be optionally substituted at one or more positions with such substituents as described herein. In some cases, the heterocycle may be bonded to a compound via a heteroatom ring atom (e.g., nitrogen). In some cases, the heterocycle may be bonded to a compound via a carbon ring atom. In some cases, the heterocycle is pyridine, imidazole, pyrazine, pyrimidine, pyridazine, acridine, acridin-9-amine, bipyridine, naphthyridine, quinoline, benzoquinoline, benzolsoquinoline, phenanthridine-1,9-diamine, or the like.

The terms “amine” and “amino” are art-recognized and refer to both unsubstituted and substituted amines, e.g., a moiety that can be represented by the general formula: N(R′)(R″)(R′″) wherein R′, R″, and R′″ each independently represent a group permitted by the rules of valence. An example of a substituted amine is benzylamine.

Any of the above groups may be optionally substituted. As used herein, the term “substituted” is contemplated to include all permissible substituents of organic compounds, “permissible” being in the context of the chemical rules of valence known to those of ordinary skill in the art. It will be understood that “substituted” also includes that the substitution results in a stable compound, e.g., which does not spontaneously undergo transformation such as by rearrangement, cyclization, elimination, etc. In some cases, “substituted” may generally refer to replacement of a hydrogen with a substituent as described herein. However, “substituted,” as used herein, does not encompass replacement and/or alteration of a key functional group by which a molecule is identified, e.g., such that the “substituted” functional group becomes, through substitution, a different functional group. For example, a “substituted phenyl group” must still comprise the phenyl moiety and can not be modified by substitution, in this definition, to become, e.g., a pyridine ring. In a broad aspect, the permissible substituents include acyclic and cyclic, branched and unbranched, carbocyclic and heterocyclic, aromatic and nonaromatic substituents of organic compounds. Illustrative substituents include, for example, those described herein. The permissible substituents can be one or more and the same or different for appropriate organic compounds. For purposes of this invention, the heteroatoms such as nitrogen may have hydrogen substituents and/or any permissible substituents of organic compounds described herein which satisfy the valencies of the heteroatoms.

Examples of substituents include, but are not limited to, halogen, azide, alkyl, aralkyl, alkenyl, alkynyl, cycloalkyl, hydroxyl, alkoxyl, amino, nitro, sulfhydryl, imino, amido, phosphonate, phosphinate, carbonyl, carboxyl, silyl, ether, alkylthio, sulfonyl, sulfonamido, ketone, aldehyde, ester, heterocyclyl, aromatic or heteroaromatic moieties, —CF₃, —CN, aryl, aryloxy, perhaloalkoxy, aralkoxy, heteroaryl, heteroaryloxy, heteroarylalkyl, heteroaralkoxy, azido, amino, halide, alkylthio, oxo, acylalkyl, carboxy esters, -carboxamido, acyloxy, aminoalkyl, alkylaminoaryl, alkylaryl, alkylaminoalkyl, alkoxyaryl, arylamino, aralkylamino, alkylsulfonyl, -carboxamidoalkylaryl, -carboxamidoaryl, hydroxyalkyl, haloalkyl, alkylaminoalkylcarboxy-, aminocarboxamidoalkyl-, cyano, alkoxyalkyl, perhaloalkyl, arylalkyloxyalkyl, and the like.

While several embodiments of the present invention have been described and illustrated herein, those of ordinary skill in the art will readily envision a variety of other means and/or structures for performing the functions and/or obtaining the results and/or one or more of the advantages described herein, and each of such variations and/or modifications is deemed to be within the scope of the present invention. More generally, those skilled in the art will readily appreciate that all parameters, dimensions, materials, and configurations described herein are meant to be exemplary and that the actual parameters, dimensions, materials, and/or configurations will depend upon the specific application or applications for which the teachings of the present invention is/are used. Those skilled in the art will recognize, or be able to ascertain using no more than routine experimentation, many equivalents to the specific embodiments of the invention described herein. It is, therefore, to be understood that the foregoing embodiments are presented by way of example only and that, within the scope of the appended claims and equivalents thereto, the invention may be practiced otherwise than as specifically described and claimed. The present invention is directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the scope of the present invention.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Other elements may optionally be present other than the elements specifically identified by the “and/or” clause, whether related or unrelated to those elements specifically identified unless clearly indicated to the contrary. Thus, as a non-limiting example, a reference to “A and/or B,” when used in conjunction with open-ended language such as “comprising” can refer, in one embodiment, to A without B (optionally including elements other than B); in another embodiment, to B without A (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

In the claims, as well as in the specification above, all transitional phrases such as “comprising,” “including,” “carrying,” “having,” “containing,” “involving,” “holding,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

What is claimed:

1. A composition, comprising:
a compound having the structure, 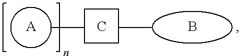
wherein:
A comprises at least one binding moiety capable of binding a target molecule;
B comprises an identification nucleotide group; and
C is a linker group tethered to A and B; and
n is at least 2,
wherein, in the presence of a target molecule, the compound can bind at least two target molecules.

2. A composition of claim 1, wherein the one binding moiety comprises a biological molecule.

3. A composition of claim 1, wherein the one binding moiety comprises a nucleophile.

4. A composition of claim 1, wherein the one binding moiety comprises an electrophile.

5. A composition of claim 1, wherein, in the presence of the target molecule, the compound can bind at least two binding sites of one or more target molecules.

6. A composition of claim 1, wherein the one binding moiety binds the target molecule via a formation of a bond.

7. A composition of claim 1, wherein the one binding moiety comprises a hydroxyl, amino, carboxyl, sulfonyl, phosphonyl, cyanate, isocyanate, epoxide, aziridine group.

8. A composition of claim 1, wherein the linker group comprises an alkyl, heteroalkyl, aryl, heteroaryl, or carboxyl group.

9. A composition of claim 1, wherein the identification nucleotide group comprises a DNA moiety.

10. A composition of claim 1, wherein the identification nucleotide group comprises a single-stranded or double-stranded DNA moiety.

11. A composition of claim 1, wherein the identification nucleotide group comprises a PCR primer sequence.

12. A composition of claim 1, wherein the identification nucleotide group comprises at least 10 bases.

13. A composition of claim 1, wherein the identification nucleotide group comprises at least 20 bases.

14. A composition of claim 1, wherein the identification nucleotide group comprises at least 30 bases.

15. A composition as claim 1, wherein the compounds interacts with at least two binding sites of one or more target molecules.

16. A composition, comprising:
a compound having the formula, 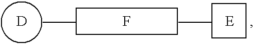
wherein:
D comprises at least one binding moiety capable of binding a first portion of a target molecule;
E comprises at least one binding moiety capable of binding or forming a bond with a second portion of the target molecule; and
F is tethered to D and E and is a linker group comprising a nucleotide group,
wherein, in the presence of the target molecule, D binds to the first portion of the target molecule and E binds to a second portion of the target molecule to form cyclic structure comprising the compound and the target molecule.

17. A composition of claim 16, wherein the one binding moiety comprises a biological molecule.

18. A composition of claim 16, wherein the one binding moiety comprises a nucleophile.

19. A composition of claim 16, wherein the one binding moiety comprises an electrophile.

20. A composition of claim 16, wherein the one binding moiety binds the target molecule via a formation of a bond.

21. A composition of claim 16, wherein the one binding moiety comprises a hydroxyl, amino, carboxyl, sulfonyl, phosphonyl, cyanate, isocyanate, epoxide, aziridine group.

22. A composition of claim 16, wherein the linker group comprises an alkyl, heteroalkyl, aryl, heteroaryl, or carboxyl group.

23. A composition of claim 16, wherein the nucleotide group comprises a DNA moiety.

24. A composition of claim 16, wherein the nucleotide group comprises a single-stranded or double-stranded DNA moiety.

25. A composition of claim 16, wherein the nucleotide group comprises a PCR primer sequence.

26. A composition of claim 16, wherein the nucleotide group comprises at least 10 bases.

27. A composition of claim 16, wherein the nucleotide group comprises at least 20 bases.

28. A composition of claim 16, wherein the nucleotide group comprises at least 30 bases.

29. A composition as in claim 16, wherein the first portion comprises a binding site of the target molecule.

30. A composition as in claim 16, wherein the second portion comprises a binding site of the target molecule.

31. A method, comprising:
providing a plurality of compounds comprising at least one active compound, each compound comprising at least one binding moiety and an identification nucleotide group tethered to the binding moiety, wherein the identification nucleotide group comprises a nucleotide sequence that is not a complement of and does not substantially interact with a nucleotide group of an adjacent compound;
exposing the plurality of compounds to a target molecule such that the at least one active compound binds the target molecule to form a bound species comprising the at least one active compound and the target molecule;
isolating the bound species; and
increasing the amount of the active compound by amplifying the identification nucleotide group of the bound species via a polymerase chain reaction.

32. A method as in claim 31, wherein the plurality of compounds comprises at least one, individual compound comprising at least two binding moieties, such that the compound binds at least two target molecules.

33. A method as in claim 31, wherein the plurality of chemical compounds comprises at least one, individual chemical compound comprising at least two binding moieties, such that the compound binds a single target molecule via each of the at least two binding moieties.

34. A method as in claim 31, wherein the act of isolating comprises exposing the bound species to a molecule comprising a nucleotide group that is a complement of the identification nucleotide group of the bound species.

35. A method as in claim 31, wherein the act of isolating comprises exposing the bound species to a molecule comprising a nucleotide group that is a full-length complement of the identification nucleotide group of the bound species.

36. A method as in claim 31, wherein the act of increasing the amount of the active compound is performed prior to the act of isolating the bound species.

37. A method as in claim 31, wherein the method comprises the act of providing at least 10² distinct, chemical compounds.

38. A method as in claim 31, wherein the method comprises the act of providing at least 10³ distinct, chemical compounds.

39. A method as in claim 31, wherein the method comprises the act of providing at least 10⁴ distinct, chemical compounds.

40. A method as in claim 31, wherein the method comprises the act of providing at least 10⁵ distinct, chemical compounds.

41. A method as in claim 31, wherein the method comprises the act of providing at least 10⁶ distinct, chemical compounds.

42. A method as in claim 31, wherein the method comprises the act of providing at least 10⁷ distinct, chemical compounds.

43. A method as in claim 31, wherein the method comprises the act of providing at least 10⁸ distinct, chemical compounds.

44. A method as in claim 31, wherein the method comprises the act of providing at least 10⁹ distinct, chemical compounds.

45. A method as in claim 31, wherein at least one active compound binds a binding site of the target molecule.
